(19) United States
(12) Patent Application Publication
(10) Pub. No.: US 2008/0200979 A1
(43) Pub. Date: Aug. 21, 2008

(54) ANTI-RESTENOTIC THERAPEUTIC DEVICE

(75) Inventors: Martin S. DIECK, Campbell, CA (US); Nicholas C. DeBEER, Montara, CA (US)

Correspondence Address: Nfocus Neuromedical Inc.,c/o Levine Bagade Han LLP, 2483 East Bayshore Road, Suite 100, Palo Alto, CA, 94303, US

(73) Assignee: NFOCUS NEUROMEDICAL, INC., Palo Alto, CA (US)

(21) Appl. No.: 12/028,726

(22) Filed: Feb. 8, 2008

Related U.S. Application Data
(63) Continuation of application No. PCT/US2006/031059, filed on Aug. 10, 2006.
(60) Provisional application No. 60/707,296, filed on Aug. 10, 2005.

(51) Int. Cl. A61F 2/06

(52) U.S. Cl. 623/1.44; 623/1.15; 623/1.17

(57) ABSTRACT

An anti-restontic device is provided for repairing a tissue, particularly an arteriosclerosed blood vessel or a damaged wall of a luminal or chambered organ. In some embodiments, the device comprises a structure having a first surface and a second surface. A bioactive layer is disposed on the first surface, wherein the bioactive layer enhances growth of a type of cells thereon. And an anti-restenosis layer is disposed on the second surface, wherein the anti-restenosis layer inhibits growth of another type of cells thereon.

### CROSS-REFERENCE TO RELATED APPLICATIONS

This application is a continuation of co-pending International Application No. PCT/US2006/031059 filed Aug. 10, 2006 which claimed benefit of priority to U.S. Provisional Application No. 60/707,296 filed Aug. 10, 2005, both applications of which are incorporated herein by reference in their entirety.

### BACKGROUND OF THE INVENTION

Vascular disease often presents as friable material on the inner lumens of blood vessels throughout the body, particularly the coronary arteries. The material accumulates over time and can eventually impede the flow of blood. When disturbed, this material can become dislodged and occlude blood flow thereby causing an ischemic event. The primary minimally invasive method of treating this disease is to open the vessel mechanically while preventing the embolic material from being dislodged causing further harm.

Two types of mechanical implants are typically used to open the vessels, stents and stent-grafts. Stents are tubes that can be delivered with the radial strength necessary to open a diseased vessel. The stents commonly have some material removed, creating open areas commonly referred to as “cells”, to improve their flexibility and make them safer and easier to deploy into the curvature of the vascular system. The material remaining provides the radial strength to reopen the vessel. However, the cells can allow the friable diseased material to extrude into the vessel lumen and may break off to cause harmful emboli. The current standard of care for using stents utilizes a separate distal protection device to catch the emboli and remove it from the body. Stent-grafts have a polymeric covering to trap potential embolic material in place and prevent the emboli from being dislodged in the first place. The polymeric covering or sleeve may have a bio-active coating that helps line the inner surface of the sleeve with endothelial cells, to provide a more blood-compatible lining in the stent-graft.

However, restenosis after percutaneous coronary intervention is a significant clinical problem, occurring after 15% to 30% of angioplasty procedures or intracoronary stenting. Such restenosis is typically due to smooth muscle cell proliferation into the stent. This is particularly problematic in saphenous vein grafts used in coronary bypass surgery. Therefore, a stent-type device is desired which promotes a more blood compatible lining yet inhibits restenosis, particularly proliferation of cells such as smooth muscle cells. At least some of these objectives will be met by the present invention.

### BRIEF SUMMARY OF THE INVENTION

An anti-restenotic device is provided for repairing a tissue, particularly an arteriosclerosed blood vessel or a damaged wall of a luminal or chambered organ. In some embodiments, the device comprises a structure having a first surface and a second surface. A bioactive layer is disposed on the first surface, wherein the bioactive layer enhances growth of a type of cells thereon. And an anti-restenosis layer is disposed on the second surface, wherein the anti-restenosis layer inhibits growth of another type of cells thereon. Thus, positioning of the structure within a blood vessel (so that the first surface faces the lumen) promotes beneficial cell growth, such as endothelial cell growth, to form a more blood-compatible lining. At the same time, the second surface inhibits ingrowth of undesirable cells which lead to restenosis, such as smooth muscle cells.

Such an anti-restontic device can also be used to repair other tissues, such as hernias, hepatic ducts, meninges, lung passageways, patent foramen ovale, atrial septal defects, and tracheal bronchial strictures, to name a few. For example, when repairing a hernia, the device may have the form of a patch which is positionable against the herniated muscle layer. The first surface which contacts the herniated muscle layer promotes beneficial cell growth, such as muscle cell ingrowth. This assists in holding the patch in place. The second surface which faces away from the herniated muscle layer inhibits growth of cells which lead to adhesions.

The anti-restenosis layer includes an anti-restenosis agent which inhibits ingrowth of undesirable cells by preventing dividing, destroying, repelling or preventing adhesion of the undesirable cells. A variety of agents may be used. Examples of such anti-restenosis agents include taxol, a pharmaceutically active taxol derivative, rapamycin, a pharmaceutically active rapamycin derivative, synthetic matrix metalloproteinase inhibitors such as batimastat (BB-94), cell-permeable mycotoxins such as cytochalasin B, gene-targeted therapeutic drugs, c-myc neutrally charged antisense oligonucleotides such as Resten-NG™, nonpeptide inhibitors such as tirofiban, antiallergic drugs such as Rizaben™ (tranilast), gene-based therapeutics such as GenStent™ biologic, heparin, paclitaxel, and any combination of these. Typically, the bioactive layer comprises a deposited layer of functional groups. And, in some embodiments, the bioactive layer further comprises a peptide coating. Aspects of these layers will be described in further detail below.

In preferred embodiments, the anti-restenosis device comprises structure comprising a tubular sleeve. In such embodiments, the first surface is typically disposed on an inner surface of the sleeve and the second surface is disposed on an outer surface of the sleeve. The structure may further comprise an expandable support frame positionable at least partially within the sleeve. The support frame provides radial force for supporting the device within a blood vessel or body lumen. Thus, the structure may act as a stent-graft for stenting blood vessels, particularly saphenous vein grafts or may be used for stenting aneurysms.

In some embodiments, the structure further comprises at least one security ring configured to secure the sleeve to the support frame. In such embodiments, the second surface may be disposed on at least one surface of the at least one security ring. Further, the first surface is typically disposed on an inner surface of the sleeve. A single security ring may be used, or multiple security rings may be spaced along the device allowing greater flexibility of the device. The rings could also align with specific portions of the underlying expandable support frame, such as alternating gaps in the internal member cells or separate sections, to provide even greater flexibility. Thus, in some embodiments, the security rings serve two purposes, to hold the sleeve in place and to deliver the anti-restenotic agent. Because of this, the rings may have very low radial strength, which would lead to a more flexible/desirable device.

The anti-restenosis layer may be disposed on the device by a variety of methods. In some embodiments, the anti-restenosis layer is disposed on the second surface by coating. In other embodiments, the anti-restenosis layer comprises a jacket positionable over at least a portion of the structure. The jacket may comprise, for example, a woven mesh, lattice, weave, tube having apertures, wrapped strand or any combination of these. Optionally, the jacket may comprise a scaffold having an anti-restenosis agent disposed thereon, wherein the scaffold comprises a polymer, metal, wire, ribbon, thread, suture, fiber, or combination of these. In other embodiments, the structure comprises a patch.

In some embodiments, the anti-restenosis device comprises a composite expandable device for assisting in maintaining patency of a blood vessel having smooth muscle cells. In such embodiments, the device includes a sleeve having an inner surface and an outer surface, and an expandable tubular support frame positionable at least partially within the sleeve. The frame is capable of expanding within the blood vessel so as to position at least a portion of the outer surface of the sleeve against a wall of the blood vessel. In such embodiments, the device also includes an anti-restenosis layer disposed on the outer surface of the sleeve, wherein the anti-restenosis layer inhibits growth of the smooth muscle cells therein when the at least a portion of the outer surface is positioned against the wall of the blood vessel.

Again, the structure further may further comprise at least one security ring configured to secure the sleeve to the support frame. The anti-restenosis layer may also be disposed on at least one surface of the at least one security ring. In any case, the device may further include a bioactive layer disposed on the inner surface of the sleeve, wherein the bioactive layer enhances growth of endothelial cells thereon.

In other embodiments, the composite expandable device comprises a sleeve having an inner surface and an outer surface, an expandable tubular support frame positionable at least partially within the sleeve, at least one security ring configured to secure the sleeve to the support frame wherein the frame is capable of expanding within the blood vessel so as to position at least one surface of the at least one security ring against a wall of the blood vessel, and an anti-restenosis layer disposed on the at least one surface of the at least one security ring, wherein the anti-restenosis layer inhibits growth of the smooth muscle cells thereon when the at least one surface of the at least one security ring is positioned against the wall of the blood vessel.

In some embodiments, the anti-restenosis layer is disposed on the at least one surface of the at least one security ring by coating. The anti-restenosis layer may comprise an anti-restenosis agent and a carrier, and in particular the anti-restenosis agent may comprise 5-30% of the layer. And, in some embodiments, the carrier is biodegradable. Again, the anti-restenosis layer may comprise a jacket positionable over at least a portion of the sleeve, wherein the jacket comprises a woven mesh, lattice, weave, tube having apertures, wrapped strand or any combination of these. Optionally, the jacket may comprise a scaffold having an anti-restenosis agent disposed thereon, wherein the scaffold comprises a polymer, metal, wire, ribbon, thread, suture, fiber or combination of these. And in some embodiments, the device further includes a bioactive layer disposed on the inner surface of the sleeve, wherein the bioactive layer enhances growth of endothelial cells thereon.

Other objects and advantages of the present invention will become apparent from the detailed description to follow, together with the accompanying drawings.

### BRIEF DESCRIPTION OF THE DRAWINGS

FIGS. 1A-1B illustrate an example embodiment of an anti-restenotic device of the present invention.

FIG. 2 illustrates an example embodiment of a composite expandable device.

FIG. 3 illustrates an embodiment of a support frame.

FIG. 4 illustrates an-embodiment of a sleeve.

FIG. 5 illustrates an embodiment of a composite expandable device having one or more security rings.

FIG. 6A illustrates a composite expandable device having portions of the support frame extending beyond the sleeve.

FIG. 6B illustrates a cross-sectional view of a portion of the device of FIG. 6A.

FIGS. 7A-7C illustrate example embodiments of jackets of the present invention.

FIGS. 8A-8B illustrate an embodiment of a composite expandable device of the present invention carried on an expandable balloon catheter.

FIGS. 9A-9C depict three combinations of jacket, sleeve and stent configuration.

It is emphasized that, according to common practice, the various features of the drawings are not to-scale. On the contrary, the dimensions of the various features are arbitrarily expanded or reduced for clarity.

### DETAILED DESCRIPTION OF THE INVENTION

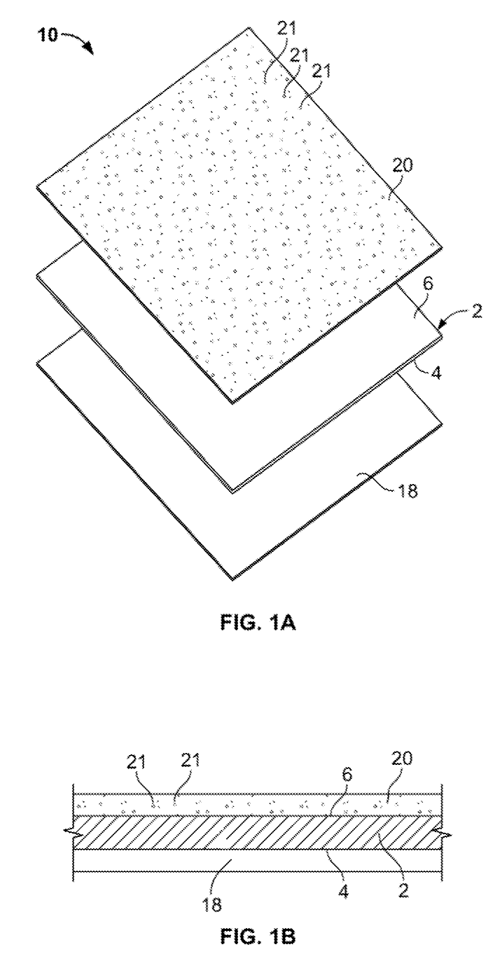

An anti-restenotic device is provided for repairing a tissue. Such a device may take a variety of forms. Examples include a patch, a sheet, a tube, a pocket, a sleeve, a stent, a graft-stent, and, particularly, a composite expandable device. FIGS. 1A-1B illustrate an example of a device 10 having the form of a patch or sheet. Here the device 10 comprises a structure 2 having a first surface 4 and a second surface 6. The device 10 further comprises a bioactive layer 18 disposed on the first surface 4, wherein the bioactive layer 18 enhances growth of a type of cells thereon. The device 10 further includes an anti-restenosis layer 20 disposed on the second surface 6, wherein the anti-restenosis layer inhibits growth of another type of cells thereon. The anti-restenosis layer includes an anti-restenosis agent 21 which is eluted therefrom as will be discussed in later sections. The structure 2 and layers 4, 6 are shown separated for illustration purposes. FIG. 1B provides a cross sectional view of the device 10 of FIG. 1A.

In some embodiments, the device also includes an impermeable layer which prevents cell migration therethrough. The impermeable layer may comprise at least a portion of the bioactive layer, at least a portion of the anti-restenosis layer, and/or a separate independent layer. The impermeable layer may be comprised of any suitable material, such as impermeable ePTFE and fluorinated ethylene propylene (FEP) to name a few.

The device 10 of FIGS. 1A-1B may be used for a variety of applications. In particular, the device 10 may be used to repair hernias, hepatic ducts, meninges, lung passageways, patent foramen ovale, atrial septal defects, and tracial bronchial strictures, to name a few. For example, when repairing a hernia, the device 10 may be positioned against the herniated muscle layer. The first surface 4 having the bio-active layer 18 contacts the herniated muscle layer promoting beneficial cell growth, such as muscle cell ingrowth. This assists in holding the patch in place. The second surface 6 having the anti-restenosis layer 20 faces away from the herniated muscle layer and inhibits growth of cells which lead to adhesions.

The device 10 may have other forms, including a composite expandable device which will be described in more detail below:

### Overview of Embodiment of Composite Expandable Device

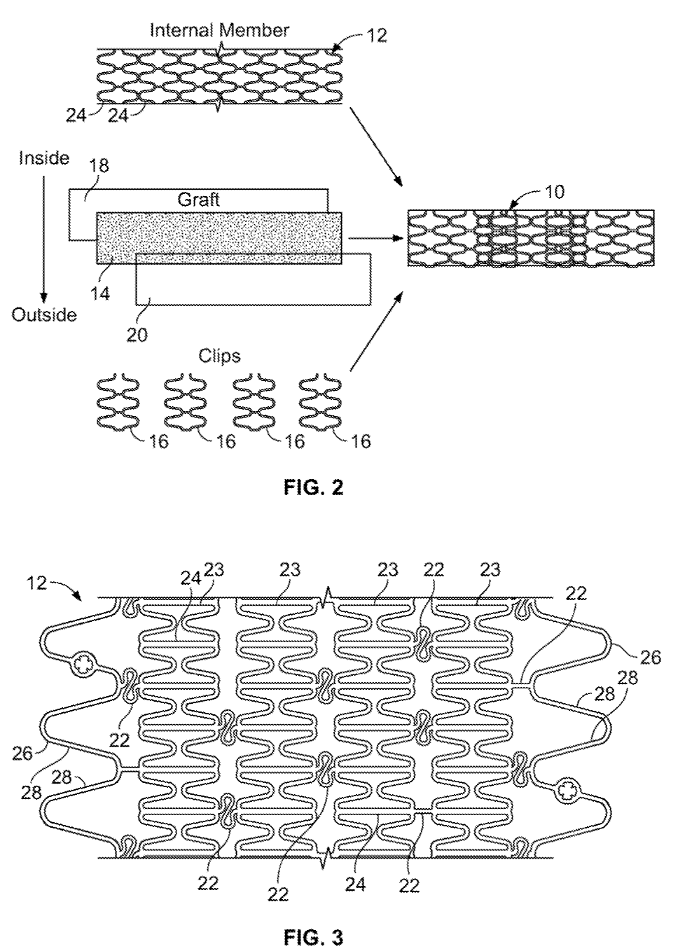

FIG. 2 illustrates an example embodiment of a composite expandable device 10 of the present invention. In this embodiment, the device 10 includes an expandable tubular support frame 12, an expandable sleeve 14 extending over the support frame 12 wherein the sleeve 14 has inner and outer surfaces (outer surface is shown), and one or more expandable clips or security rings 16 which assist in anchoring the sleeve 14 to the support frame 12.

In this embodiment, the expandable sleeve 14 includes a bioactive layer 18, disposed on its inner surface. The inner surface of the sleeve 14 is treated to functionalize the sleeve material with chemical functional groups, such as hydroxyl, acid, or amine groups, for attaching coatings. Following this treatment, the inner surface of the sleeve 14 may be further treated to introduce chemical spacers and/or bioactive molecules. When the device 10 is positioned within a blood vessel, this functionalized inner surface of the sleeve 14 will be in contact with blood. The interaction of the blood with the functionalized surface forms a biocompatible layer. This layer is effective in promoting a layer of endothelial cells on the inner surface of the sleeve 14 to mimic the endothelial-cell lining of a normal vessel, making the device more compatible to blood cells flowing through the device 10.

In this embodiment, the device 10 also includes an anti-restenosis layer 20 on the outer surface of the sleeve 14. The layer 20 is comprised of an anti-restenosis drug, compound or agent that is attached to the surface, either alone or in combination with a carrier. Optionally, the outer surface of the sleeve 14 may be treated to functionalize the sleeve material with chemical functional groups, such as hydroxyl, acid, or amine groups, for assisting in attaching the layer 20. Thus, the outer surface of the device 10 releases an anti-restenosis agent into the walls of the blood vessel in contact with the device 10, reducing the risk of hyper-proliferation and restenosis of the blood vessel.

Thus, the bioactive layer 18 on the inner surface of the sleeve 14 and the anti-restenosis layer 20 on the outer surface of the sleeve 14 combine to provide an expandable device 10 having superior patency properties. The bioactive layer 18 acts to promote endothelial-cell growth at the inner graft surface, making the device more compatible to blood cells flowing through the stent graft. At the same time, anti-restenosis layer 20 releases the anti-restenosis agent into the blood vessel surfaces in contact with the device, reducing the risk of hyper-proliferation and restenosis of the blood vessel. The device 10 thus acts to reinforce the walls of the blood vessel, produce a blood-compatible lining therein, and reduce the risk of re-occluding by hyper-proliferation of the blood vessel wall cells, in response to the mechanical injury caused by placement of the stent graft.

The composite expandable device 10 of the present invention may have a variety of forms and combinations of features. Examples of such features are described in more detail below. It may be appreciated that such features may be combined in any combination.

### Support Frame

The support frame 12 may have a variety of forms. The frame 12 is expandable from a contracted, small-diameter condition to a radially expanded condition under the influence of an expanding force, typically an expandable balloon catheter used in delivering and placing the device in a blood vessel, according to conventional stent placement methods.

An exemplary support frame 12 comprises a stent described in U.S. Pat. No. 6,371,980, filed Aug. 30, 1999 and issued Apr. 16, 2002, which is incorporated by reference herein in its entirety. An example of such a support frame 12 is illustrated in FIG. 3. As shown, the frame 12 has of a plurality of axially spaced-apart circular belts 23 which are interconnected by interconnectors 22. Each belt 23 is comprised of a plurality of circumferentially spaced-apart elongate struts 24. The interconnectors 22 adjoin the ends of the struts 24 and form in conjunction therewith the circular belts 21. The interconnectors 22 are disposed at circumferentially spaced-apart positions to provide circumferential support when the stent is expanded while at the same time being axially flexible. In preferred embodiments, the interconnectors 22 are sinusoidal or serpentined shaped which assist in allowing expansion.

The frame 12 of FIG. 3 also includes two end belts 26. The end belts 26 are also connected with the remaining belts 23 by interconnectors 22. Each end belt 26 includes a plurality of circumferentially spaced-apart elongate struts 28. The interconnectors 22 allow the belts 23 and the end belts 26 to extend along an axis while permitting axial bending between the belts 23 and the end belts 26. Thus, with the construction shown in FIG. 3, there are provided four belts 23 and two end belts 26 with five sets of interconnecting elements 22. The number of belts and interconnecting elements may vary depending on the desired length of the frame 12.

The frame 12 may be formed a tube having a desired pattern formed or cut therefrom, such as by laser cutting or chemical etching. Alternatively, the desired pattern may be formed out of a flat sheet, e.g. by laser cutting or chemical etching, and then rolling that flat sheet into a tube and joining the edges, e.g. by welding. Further, the frame 12 may be formed by etching a pattern into a material or mold and depositing stent material in the pattern, such as by chemical vapor deposition or the like. Any other suitable manufacturing method known in the art may be employed for manufacturing a stent in accordance with the invention.

The frame 12 may be comprised of plastic, metal or other materials and may exhibit a multitude of configurations. Example plastics include polyurethanes and polycarbonates. Example metals include stainless steel, titanium, Nitinol, and tantalum among others.

It may be appreciated that the frame 12 may have a variety of other forms, including conventional stents, coils, wireframes, etc.

### Sleeve

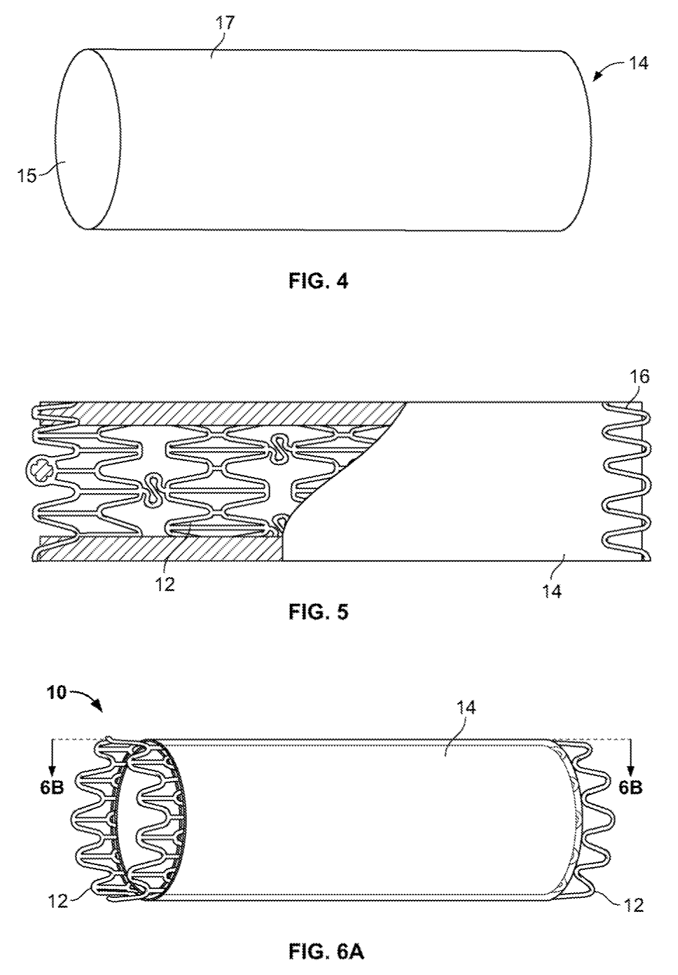

FIG. 4 illustrates an embodiment of a sleeve 14 of the present invention. Here, the sleeve 14 has a tubular shape having an inner surface 15 and an outer surface 17. The sleeve 14 is typically configured for fitting over the support frame 12, however the sleeve 14 may alternatively be disposed under the frame 12 and attached thereto. Thus, the sleeve 14 is also expandable from a contracted, small-diameter condition to a radially expanded condition. This may be achieved by constructing the sleeve 14 from a flexible material, such as a polymer. Example materials include expandable polymer material, e.g., a porous or non-porous polytetrafluoroethylene (PTFE) material.

An exemplary sleeve 14 is described in U.S. Pat. No. 6,371,980, issued Apr. 16, 2002, which is incorporated by reference herein in its entirety. It may be appreciated that the sleeve 14 may have a variety of other forms, including conventional sleeves, spirals or helixes.

### Security Rings

In some embodiments, the device 10 includes one or more clips or security rings 16 which are used to secure the sleeve 14 to the underlying frame 12, as illustrated in FIG. 5. Exemplary security rings 16 are described in U.S. patent application Ser. No. 10/255,199, filed Sep. 26, 2002, incorporated herein by reference for all purposes. In order to ensure that the sleeve 14 remains in the desired position on the frame 12, security rings 16 are positioned over the sleeve 14, such as over the outer ends of the sleeve 14. The security rings 16 may be formed of a metal and preferably the same metal which is used for the frame 12, for example, stainless steel or titanium or alloys thereof. Or, the rings 16 may be comprised of other suitable material, such as a polymer. By way of example, the security rings 16 can be formed from laser cut tubing in the same manner as some embodiments of the frame 12 having a suitable wall thickness of 0.003″ to 0.006″. The inner surfaces of the security rings 16 can be left unpolished so that they have a rougher inner surface finish to enhance gripping to the outer surface of the sleeve 14. Alternatively, a texture can be applied to the inner surface to enhance the gripping capabilities of the security ring 16.

The rings 16 may have a variety of shapes, including sinusoidal-shaped convolutions so that they can be expanded with the frame 12 and sleeve 14. The security rings 16 can be placed at any location along the device 10. In preferred embodiments, a ring 16 is used to fasten an end portion of the sleeve 14 over the confronting end portion of the frame 12, wherein the ring 16 is crimped to secure the sleeve 14 to the frame 12. The ring 16 can then be expanded in a manner similar to the frame 12. Additional rings 16 may also be employed, being placed at positions intermediate to the two end clips or security rings along the length of the device 10 as indicated in FIG. 2. Optionally, the rings 16 may also include at least one radiopaque marker.

It may be appreciated that other structures may be employed in the device 10 for anchoring the sleeve 14 on the structural frame 12. For example, the sleeve 14 could be sewn on the frame 12 or bonded to the frame 12 by polymer welds or the like.

### Bioactive Layer

In some embodiments, the device 10 includes a biomimetic or bioactive layer 18. Typically, the bioactive layer 18 is disposed on the inner surface 15 of the sleeve 14 and will therefore be described as an example. However, it may be appreciated that the layer 18 may alternatively or in addition be disposed on the outer surface 17 of the sleeve 14, or any other surface of the device 10.

In order to provide a cell-friendly bioactive layer 18 the surface 15 may be treated in the manner described in U.S. patent application Ser. No. 09/385,692 filed Aug. 30, 1999 and WO 03/070125A1 filed Dec. 21, 2001, both incorporated herein by reference for all purposes. Thus the surface 15 of the sleeve 14 can be characterized as having applied thereto a bioactive coating or layer which is cell friendly and which enhances growth of cells thereon. As described therein, a low temperature plasma-deposited layer is provided on the surface of the sleeve 14 to functionalize the surface and provide chemical functional groups, such as hydroxyl, acid, or amine groups. A spacer/linker molecular layer is covalently bonded to the plasma-deposited layer. A peptide coating such as P15 (Gly-Thr-Pro-Gly-Pro-Gln-Gly-Ile-Ala-Gly-Gln-Arg-Gly-Val-Val; SEQ ID NO: 1) is deposited on the spacer/linker layer. Together, these layers form the bioactive layer 18.

When the device 10 is positioned within a blood vessel, this functionalized inner surface 15 of the sleeve 14 will be in contact with blood. The interaction of the blood with the functionalized surface forms a biocompatible and biomimetic layer. This layer is effective in promoting a layer of endothelial cells on the inner surface of the sleeve 14 to mimic the endothelial-cell lining of a normal vessel, making the device more compatible to blood cells flowing through the device 10.

### Anti-Restenosis Layer

The anti-restenosis layer 20 is typically comprised of an anti-restenosis agent, an anti-restenosis agent combined with a carrier, a separate scaffold supporting the anti-restenosis agent and/or the carrier, or any combination of these. Example anti-restenosis agents include taxol and its active congeners and analogs, and rapamycin and its active congeners and analogs. Other examples include synthetic matrix metalloproteinase inhibitors such as batimastat (BB-94), cell-permeable mycotoxins such as cytochalasin B, gene-targeted therapeutic drugs, c-myc neutrally charged antisense oligonucleotides such as Resten-NG™, nonpeptide inhibitors such as tirofiban, antiallergic drugs such as Rizaben™ (tranilast), gene-based therapeutics such as GenStent™ biologic, heparin, paclitaxel, and any combination of these. The amount of anti-restenosis agent in the anti-restenosis layer 20 is selected to provide a therapeutic amount of agent when released over an extended period of time, such as several days to several weeks.

The anti-restenosis layer 20 may take a variety of forms. The following forms are provided for purposes of example but are not so limited.

1) Coating of Sleeve with Anti-restenosis Layer

In some embodiments, the sleeve 14 is coated with the anti-restenosis layer 20. Typically, the outer surface 17 of the sleeve 14 is coated, but other surfaces (such as the inner surface 15) may be coated alternatively or in addition.

In some embodiments, the anti-restenosis agent is disposed in a carrier, such as a polymer, to form an agent-carrier composition. The carrier may be biodegradable or non-biodegradable. Example carriers include polymers, polyimides, poly-butyl methacrylate (PBMA), poly-butadiene (PBD), glycolide, lactide, E-caprolactone, and polyethylene glycol, poly(ester-amide) (PEA) homologs, and combinations of these, to name a few. And, the agent-carrier composition typically includes anti-restenosis agent in an amount between 5-30% of the total coating material, however other ratios may be used. The agent-carrier composition is then applied to the surface 17 of the sleeve 14 by a suitable method, such as spraying, painting, or dipping, to name a few. Optionally, the outer surface 17 of the sleeve 14 may be plasma treated and functionalized, as above, to provide a bonding surface for covalent or entangled-polymer attachment of the carrier to the outer surface 17. Typically the agent-carrier composition is applied to form a thin coating, such as a final dry thickness of between 20-50 microns.

In other embodiments, the anti-restenosis agent is coated on the outer surface 17 of the sleeve 14 as a non-polymer coating formed of the anti-restenosis agent alone or the agent in combination with non-polymer binding agents, such as are known in the art. In such embodiments, the sleeve 14 is preferably comprised of, but not limited to, a porous polymer material, e.g., porous PTFE, whose pores provide an anchoring surface for the anti-restenosis agent coating. Additionally or alternatively, the outer surface 17 may be plasma treated and optionally, further functionalized and then derivatized with strands of polymers, e.g., polyethylene glycol. The strands of polymers embedded in the dried-anti-restenosis agent coating act to anchor to the coating to the sleeve 14. As above, the anti-restenosis agent typically forms a thin coating, such as a final dry thickness of between 20-50 microns.

Devices 10 having a sleeve 14 coated with the anti-restenosis layer 20 provide many beneficial features, particularly in comparison to conventional drug-eluting stents. Conventional drug-eluting stents comprise a stent structure which supports the drug that elutes therefrom. Therefore, the drug is delivered to the blood vessel wall in locations that contact the stent structure itself. Thus, the larger the cell geometry or the more the physician expands the stent, the further apart the drug delivery locations along the blood vessel wall. This may leave “cold spots” along the blood vessel wall that receive less drug delivery. Further, the amount and arrangement of drug delivery is limited by the geometry of the stent.

By providing a composite expandable device 10 having a sleeve 14 coated with an anti-restenosis layer 20, delivery of the anti-restenosis agent is controlled, maximized and not limited by the geometry of the support frame 12. Therefore, support frames 12 having a more open cell geometry may be used. This may enhance flexibility of the frame 12 allowing delivery to more locations within the vasculature, such as through tortuous blood vessels.

2) Coating of Security Rings with Anti-restenosis Layer

In some embodiments, the one or more surfaces of one or more security rings 16 are coated with the anti-restenosis layer 20. Typically, a surface is coated that will contact the blood vessel wall. This may assist in transfer of the anti-restenosis agent to the blood vessel. Alternatively or in addition, other surfaces of the security rings 16 may be coated.

Such coating may be similar to the coatings and methods of application described above in relation to coating the sleeve 14. For example, the anti-restenosis agent may be disposed in a carrier, such as a polymer, to form an agent-carrier composition. The carrier may be biodegradable or non-biodegradable. Example carriers include polymers, polyimides, poly-butyl methacrylate (PBMA), poly-butadiene (PBD), glycolide, lactide, E-caprolactone, and polyethylene glycol, poly(ester-amide) (PEA) homologs, and combinations of these, to name a few. And, the agent-carrier composition typically includes anti-restenosis agent in an amount between 5-30% of the total coating material, however other ratios may be used. The agent-carrier composition is then applied to a surface of the security ring 16 by a suitable method, such as spraying, painting, or dipping, to name a few. After the agent-carrier composition is applied in liquid form to the security rings 16, the rings 16 are allowed to dry to form a stable coating on each ring 16, either before but typically after attachment of the rings 16 to the device 10.

Upon expansion of the composite expandable device 10, the anti-restenosis rings 16 having anti-restenosis agent thereon are brought into contact with the blood vessel wall. Thus, the anti-restenosis agent is released to the blood vessel, preferably over an extended time period, such as at least 2-3 days and up to 2 or more weeks after placement of the device 10 in the blood vessel.

In an alternative embodiment, the anti-restenosis agent is applied to the security rings 16 as a solution and allowed to dry, forming a polymer-free anti-restenosis coating on the rings 16. Adherence of the anti-restenosis coating to the rings 16 may be enhanced by roughening the surfaces of the rings 16, according to known methods. Again, upon expansion of the composite expandable device 10, the security rings 16 having anti-restenosis agent thereon are brought into contact with the blood vessel wall. The anti-restenosis agent is released to the blood vessel, however such release may be quicker than when a carrier is used.

By varying the number of security rings 16 coated with anti-restenosis agent, varying the surfaces of such rings 16 coated, and varying the placement of the security rings 16 along the device 10, the amount and pattern of agent delivery may be controlled. In addition, the coated security rings 16 may be used in combination with a sleeve 14 having coated surfaces. This may be particularly useful in providing uninterrupted agent delivery along the length of the device 10. Further, different surfaces may be coated with different types of anti-restenosis agents for a combination effect. It may be appreciated that any combination of coated surfaces may be used.

3) Coating of Support Frame with Anti-restenosis Layer

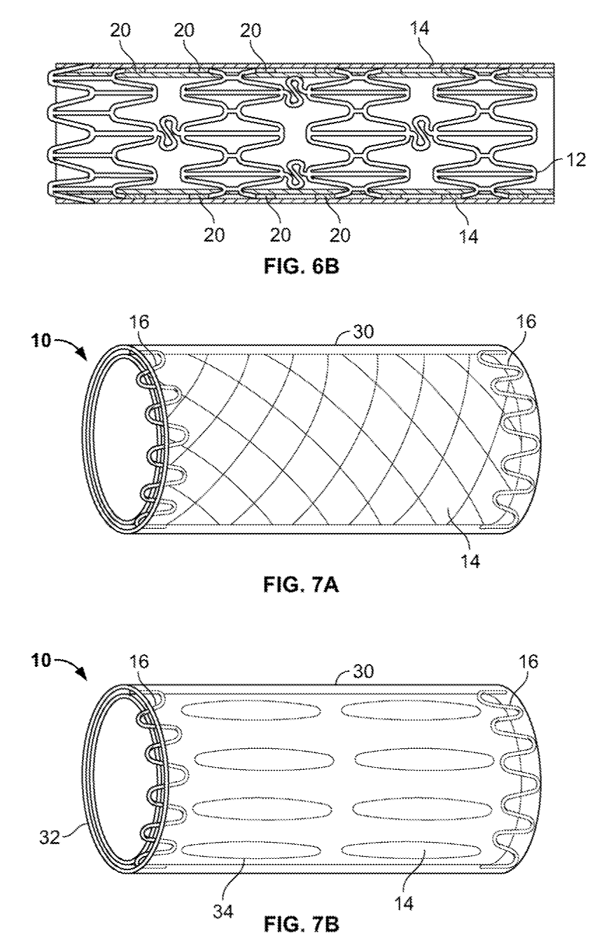

In some embodiments, the one or more surfaces of the support frame 12 are coated with the anti-restenosis layer 20. As shown in FIG. 6A, portions of the support frame 12 may extend beyond the sleeve 14 and therefore contact the blood vessel wall when the device 10 is expanded therein. Thus, coating of such surfaces of the support frame 12 deliver anti-restenosis agent directly to the blood vessel wall.

FIG. 6B illustrates a cross-sectional view of a portion of the device of FIG. 6A. As shown, the support frame 12 disposed within the sleeve 14 contacts the sleeve 14 at various locations. An anti-restenosis layer 20 coating the support frame 12 will contact the sleeve 14 at these same locations, as shown. Thus, the anti-restenosis layer 20 can be used to bond or secure the support frame 12 to the sleeve 14 at these locations. In such embodiments, the anti-restenosis layer 20 comprises an anti-restenosis agent and a curable carrier. The frame 12 is coated with the anti-restenosis layer 20 and assembled with the sleeve 14. The carrier is then cured, fixing the sleeve 14 to the frame 12 at various contacting locations. Upon delivery, the support frame 12 will expand along with the sleeve 14 secured thereto. Therefore, such embodiments may not utilize security rings 16. After the device 10 is implanted, the anti-restenosis agent is eluted from anti-restenosis layer 20 to the blood vessel. Optionally, the anti-restenosis layer 20 may be biodegradable over time. In such instances, the sleeve 14 will be held in place by the expanded support frame 12.

### 4) Jacket as Anti-restenosis Layer

In some embodiments, the anti-restenosis layer 20 comprises a jacket that is positionable over at least a portion of the composite expandable device 10. The jacket may be held in place by one or more security rings 16, or the jacket may cover the security rings 16. Further, in some embodiments, the jacket is disposed at least partially within the device 10.

The jacket may be formed from an agent-carrier composition wherein the anti-restenosis agent elutes therefrom. In such embodiments, the carrier may be biodegradable or non-biodegradable. Or, the jacket may be formed from a scaffold having an agent or an agent-carrier composition disposed thereon, such as by coating. In such embodiments, the scaffold and/or carrier may be biodegradable or non-biodegradable.

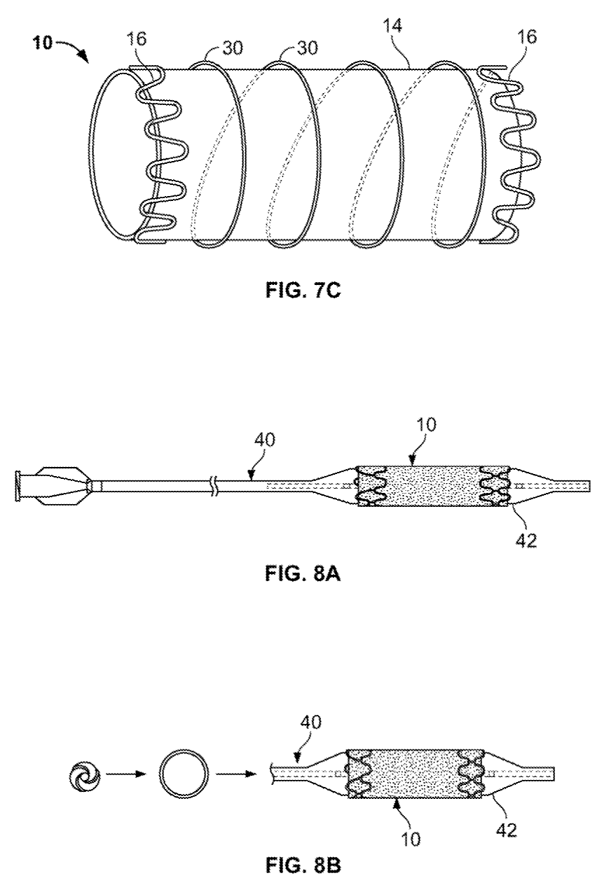

FIGS. 7A-7C illustrate example embodiments of jackets 30 of the present invention. FIG. 7A illustrates a jacket 30 comprising a woven mesh, lattice, weave. Such a jacket may be comprised of the agent-carrier composition itself or of a scaffold having the anti-restenosis agent thereon, as described above. Such scaffolds may be comprised of polymer strands, metal wire or ribbon, thread, suture, or fibers, to name a few. FIG. 7B illustrates a jacket 30 comprising a tube 32 having cutouts or apertures 34. Such apertures 34 may be any size or shape and may be of any number or arrangement. Again, such a jacket may be comprised of the agent-carrier composition itself or of a scaffold having the anti-restenosis agent thereon, as described above. Such scaffolds may be comprised of, for example, polymers or metals, particularly laser cut tubes. FIG. 7C illustrates a jacket 30 comprising a strand wrapped around the device 10, such as in a coiled fashion. Such a jacket may be comprised of the agent-carrier composition itself or of a scaffold having the anti-restenosis agent thereon, as described above. Such scaffolds may be comprised of polymer strands, metal wire or ribbon, thread, suture, or fibers, to name a few.

Such jackets 30 may provide a more easily manufacturable anti-restenosis layer 20. Alternatively or in addition, such jackets 30 may allow a more even distribution of anti-restenosis agent and elution therefrom.

### 5) Security Rings as Anti-restenosis Layer

In some embodiments, the anti-restenosis layer 20 acts as a security ring 16. In such embodiments, the security ring 16 may be formed from the agent-carrier composition itself wherein the anti-restenosis agent elutes therefrom. In such embodiments, the carrier may be biodegradable or non-biodegradable.

### Delivery

FIGS. 8A-8B illustrate an embodiment of a composite expandable device 10 of the invention carried on an expandable balloon catheter 40 for deployment within a blood vessel. The composite expandable device 10 is carried on the distal end of the balloon catheter 40, such as by crimping the device 10 over the balloon 42. Once positioned within a blood vessel, the balloon 42 is expanded which expands the composite expandable device 10 until the outer surfaces of the device 10 are brought into contact with the wall of the blood vessel.

Once positioned in the blood vessel, the bioactive layer 18 that may be carried on the inner surface 15 of the sleeve 14 acts to promote endothelial-cell growth at the inner surface 15, making the device 10 more compatible to blood cells therethrough. At the same time, the anti-restenosis layer 20 begins to release anti-restenosis agent into the blood vessel, reducing the risk of hyper-proliferation and restenosis of the blood vessel.

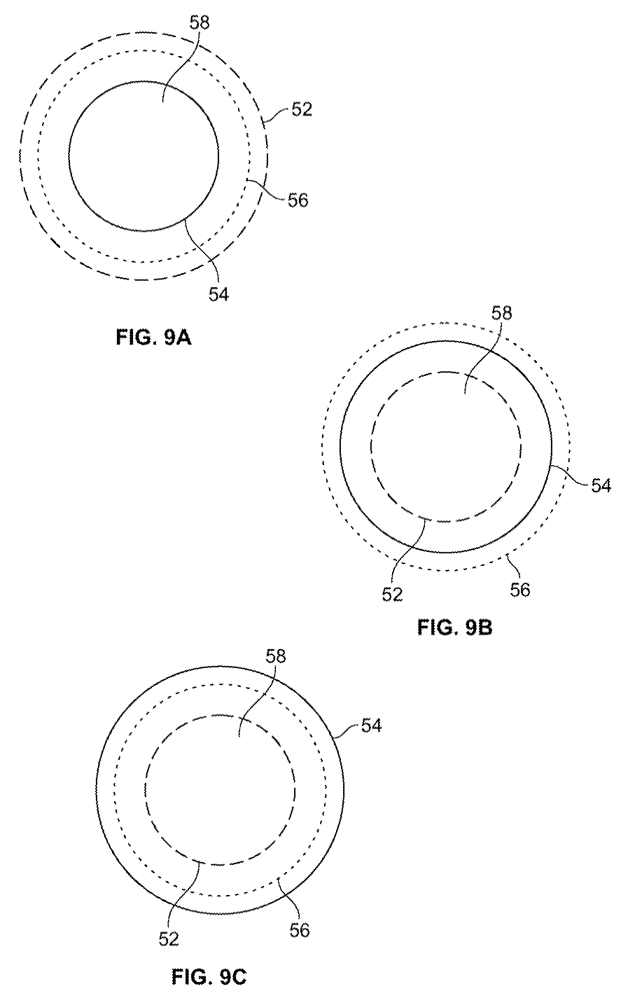

The composite expandable device 10 thus acts to reinforce the walls of the blood vessel, produce a blood-compatible lining therein, and reduce the risk of re-occluding by hyper-proliferation of the blood vessel wall cells, in response to any possible mechanical injury that may be caused by placement of the device 10. FIGS. 9A-9C depict jacket 52, sleeve 56, and stent 54 within lumen 58.

It will be appreciated that embodiments described with respect to one aspect may be applicable to each aspect of the compositions and methods described. It will further be appreciated that embodiments may be used in combination or separately. It will also be realized that sub-combinations of the embodiments may be used with the different aspects. Although the embodiments have been described with many optional features, these features are not required unless specifically stated.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. An anti-restenotic device for repairing a tissue comprising: a structure having a first surface and a second surface; a bioactive layer disposed on the first surface, wherein the bioactive layer enhances growth of a type of cells thereon; and an anti-restenosis layer disposed on the second surface, wherein the anti-restenosis layer inhibits growth of another type of cells thereon.

2. The device according to claim 1, wherein the anti-restenosis layer includes an anti-restenosis agent selected from the group consisting of taxol, a pharmaceutically active taxol derivative, rapamycin, a pharmaceutically active rapamycin derivative, and any combination of these.

3. The device according to claim 1 or 2, wherein the bioactive layer comprises a deposited layer of functional groups.

4. The device according to claim 3, wherein the bioactive layer further comprises a peptide coating.

5. The device according to any previous claim, wherein the structure comprises a tubular sleeve.

6. The device according to claim 5, wherein the first surface is disposed on an inner surface of the sleeve and the second surface is disposed on an outer surface of the sleeve.

7. The device according to claim 5, wherein the structure further comprises an expandable support frame positionable at least partially within the sleeve.

8. The device according to claim 7, wherein the structure further comprises at least one security ring configured to secure the sleeve to the support frame.

9. The device according to claim 8, wherein the second surface is disposed on at least one surface of the at least one security ring.

10. The device according to claim 9, wherein the first surface is disposed on an inner surface of the sleeve.

11. The device according to any previous claim, wherein the anti-restenosis layer is disposed on the second surface by coating.

12. The device according to any previous claim, wherein the anti-restenosis layer comprises a jacket positionable over at least a portion of the structure, wherein the jacket comprises a woven mesh, lattice, weave, tube having apertures, wrapped strand or any combination of these.

13. The device according to claim 12, wherein the jacket comprises a scaffold having an anti-restenosis agent disposed thereon, wherein the scaffold comprises a polymer, metal, wire, ribbon, thread, suture, fiber, or combination of these.

14. The device according to any previous claim, wherein at least a portion of the anti-restenosis layer is biodegradable.

15. The device according to any previous claim, wherein the structure comprises a patch.

16. A composite expandable device for assisting in maintaining patency of a blood vessel having smooth muscle cells, the device comprising: a sleeve having an inner surface and an outer surface; an expandable tubular support frame positionable at least partially within the sleeve, wherein the frame is capable of expanding within the blood vessel so as to position at least a portion of the outer surface of the sleeve against a wall of the blood vessel; an anti-restenosis layer disposed on the outer surface of the sleeve, wherein the anti-restenosis layer inhibits growth of the smooth muscle cells therein when the at least a portion of the outer surface is positioned against the wall of the blood vessel.

17. The device according to claim 16, wherein the anti-restenosis layer includes an anti-restenosis agent selected from the group consisting of taxol, a pharmaceutically active taxol derivative, rapamycin, a pharmaceutically active rapamycin derivative, and any combination of these.

18. The device according to claim 16 or 17, wherein the structure further comprises at least one security ring configured to secure the sleeve to the support frame.

19. The device according to claim 18, wherein the anti-restenosis layer is also disposed on at least one surface of the at least one security ring.

20. The device according to any one of claims 16 to 19, further comprising a bioactive layer disposed on the inner surface of the sleeve, wherein the bioactive layer enhances growth of endothelial cells thereon.

21. The device of claim 16, further comprising at least one security ring configured to secure the sleeve to the support frame wherein the frame is capable of expanding within the blood vessel so as to position at least one surface of the at least one security ring against a wall of the blood vessel.

22. The device according to claim 21, wherein the anti-restenosis layer includes an anti-restenosis agent selected from the group consisting of taxol, a pharmaceutically active taxol derivative, rapamycin, a pharmaceutically active rapamycin derivative, and any combination of these.

23. The device according to claim 21 or 22, wherein the anti-restenosis layer is disposed on the at least one surface of the at least one security ring by coating.

24. The device according to claim 23, wherein the anti-restenosis layer comprises an anti-restenosis agent and a carrier and wherein the anti-restenosis agent comprises 5-30% of the layer.

25. The device according to claim 23, wherein the anti-restenosis layer comprises an anti-restenosis agent and a carrier and wherein the carrier is biodegradable.

26. The device according to any one of claims 21 to 25, wherein the anti-restenosis layer comprises a jacket positionable over at least a portion of the sleeve, wherein the jacket comprises a woven mesh, lattice, weave, tube having apertures, wrapped strand or any combination of these.

27. The device according to claim 26, wherein the jacket comprises a scaffold having an anti-restenosis agent disposed thereon, wherein the scaffold comprises a polymer, metal, wire, ribbon, thread, suture, fiber or combination of these.

28. The device according to any one of claims 21 to 27, further comprising a bioactive layer disposed on the inner surface of the sleeve, wherein the bioactive layer enhances growth of endothelial cells thereon.

29. A stent-graft complex comprising, an expandable tubular lattice support structure, a graft disposed adjacent the support structure, and a scaffolding jacket disposed adjacent a member selected from the graft and the support structure.

30. The stent-graft complex of claim 29, the scaffolding jacket selected from a woven mesh, a lattice, a weave, polymer strands, metal wire, ribbon, thread, suture, fibers, a wrapped strand, and a coiled strand.

31. The stent-graft complex of claim 29, the scaffolding jacket comprising an agent-carrier composition capable of releasing agent from the scaffold.

32. The stent-graft complex of claim 29, the scaffolding jacket comprising a biodegradable material.

33. The stent-graft complex of claim 29, the graft disposed on an inside of the support structure.

34. The stent-graft complex of claim 29, the graft disposed on an outside of the support structure.

35. The stent-graft complex of claim 29, the jacket disposed on an inside of the graft.

36. The stent-graft complex of claim 29, the jacket disposed on an outside of the graft.

37. The stent-graft complex of claim 29, the jacket disposed on an inside of the support structure.

38. The stent-graft complex of claim 29, the jacket disposed on an outside of the support structure.

39. The stent-graft complex of claim 29, the expandable tubular lattice support structure being balloon expandable.

40. The stent-graft complex of claim 29, further comprising a bioactive layer disposed on the graft.

41. The stent-graft complex of claim 40, the bioactive layer disposed on an inner surface of the graft.

42. The stent-graft complex of claim 40, the bioactive layer disposed on an outer surface of the graft.

43. The stent-graft complex of claim 41, the bioactive layer effective in promoting a layer of endothelial cells on the inner surface of the sleeve to mimic the endothelial lining of a normal vessel.

44. The stent-graft complex of claim 29, further comprising an impermeable layer which prevents cell migration therethrough.

45. A method of treating a vessel comprising:
placing in the vessel an expandable tubular lattice support structure having a graft disposed adjacent the support structure, and
positioning a scaffolding jacket adjacent a member selected from the graft and the support structure, thereby promoting tissue endothelization in the vessel.

46. The method of claim 45, the scaffolding jacket selected from a woven mesh, a lattice, a weave, polymer strands, metal wire, ribbon, thread, suture, fibers, a wrapped strand, and a coiled strand.

47. The method of claim 45, the scaffolding jacket comprising an agent-carrier composition.

48. The method of claim 45, the scaffolding jacket comprising a biodegradable material.

49. The method of claim 45, the graft comprising a bioactive layer on the graft, the bioactive layer capable of promoting endothelialization.

50. A method of deploying a stent-graft comprising:
positioning an expandable tubular lattice support structure,
disposing a graft adjacent the support structure forming a stent-graft, and
positioning a scaffolding jacket adjacent the stent graft.

51. The method of claim 50, the scaffolding jacket selected from a woven mesh, a lattice, a weave, polymer strands, metal wire, ribbon, thread, suture, fibers, a wrapped strand, and a coiled strand.

52. The method of claim 50, the scaffolding jacket comprising an agent-carrier composition.

53. The method of claim 50, the scaffolding jacket comprising a biodegradable material.

54. The method of claim 50, the graft comprising a bioactive layer on the graft, the bioactive layer capable of promoting endothelization.
